(19) United States
(12) Patent Application Publication
(10) Pub. No.: US 2025/0049502 A1
(43) Pub. Date: Feb. 13, 2025

(54) REDUNDANT POWER SENSING FOR ABLATION SYSTEMS

(71) Applicant: INTUITIVE SURGICAL OPERATIONS, INC., Sunnyvale, CA (US)

(72) Inventors: Andrey Polonsky, Rochester, NY (US); Kyle Locke, Atascadero, CA (US); Dong Hao Geng, Fremont, CA (US)

(21) Appl. No.: 18/797,624

(22) Filed: Aug. 8, 2024

Related U.S. Application Data
(60) Provisional application No. 63/518,416, filed on Aug. 9, 2023.

(51) Int. Cl. A61B 18/18; A61B 18/00; G01R 21/02

(52) U.S. Cl. CPC A61B 18/1815; G01R 21/02; A61B 2018/00577; A61B 2018/00702; A61B 2018/00785; A61B 2018/00791; A61B 2018/00827; A61B 2018/00845; A61B 2018/00892; A61B 2018/00982; A61B 2018/1823

(57) ABSTRACT

Methods and systems for redundant power detection, including forward power and reverse power, utilize sensors separate from microwave power sensors. The redundant power detection can be compared to measurements of the microwave power sensors to ensure that the sensors are operating correctly.

### CROSS-REFERENCE TO RELATED APPLICATION

This application claims the priority benefit of U.S. Provisional Application No. 63/518,416, filed Aug. 9, 2023, which is hereby incorporated by reference herein in its entirety.

### FIELD

Disclosed embodiments relate to ablation systems.

### BACKGROUND

Ablation instruments transmit energy in the form of electromagnetic waves to a targeted area of tissue, such as a tumor or other growth, within the patient anatomy to treat (e.g., destroy) the targeted tissue. Some ablation instruments may be delivered to target tissue using minimally invasive techniques. For example, as flexible and/or steerable elongate device, such as a flexible catheter or endoscope, can be inserted into anatomic passageways and navigated toward a region of interest within the patient anatomy to deliver the ablation instrument. Various features may improve the effectiveness of such ablation instruments.

### SUMMARY

The following presents a simplified summary of various examples described herein and is not intended to identify key or critical elements or to delineate the scope of the claims.

In some examples, a microwave energy system is disclosed that includes a microwave energy source receiving power supplied from a power supply, an ablation probe coupled to the microwave energy source to receive a forward power from the microwave energy source, and a control system. The control system is configured to measure the power supplied to the microwave energy source by the power supply, determine lost power due to inefficiency of the microwave energy source, and determine the forward power from the microwave energy source by subtracting the lost power from the power supplied to the microwave energy source.

In some examples, a method for power detection in an ablation microwave energy system is disclosed that includes measuring power supplied to a microwave energy source by a power supply, determining lost power due to inefficiency of the microwave energy source, and determining forward power sent to an ablation probe from the microwave energy source by subtracting the lost power from the power supplied to the microwave energy source.

In some examples, a non-transitory computer readable medium having instructions stored thereon is disclosed. The instructions, when executed by a computing device, cause the computing device to measure power supplied to a microwave energy source by a power supply, determine lost power due to inefficiency of the microwave energy source, and determine forward power sent to an ablation probe from the microwave energy source by subtracting the lost power from the power supplied to the microwave energy source.

In some examples, a microwave energy system is disclosed that includes a microwave energy source having a forward power heat source and a termination point for reverse power, an ablation probe coupled to the microwave energy source to receive a forward power therefrom, and a control system. The control system is configured to determine a first temperature associated with the forward power heat source, determine a second temperature associated with the termination point, and determine reverse power based on a temperature difference between the first and second temperatures.

In some examples, a method for power detection in an ablation microwave energy system is disclosed that includes determining a first temperature associated with a forward power heat source of a microwave energy source with a first temperature sensor, determining a second temperature associated with a termination point of the reverse microwave power with a second temperature sensor, and determining reverse power based on a temperature difference between the first and second temperatures.

In some examples, a non-transitory computer readable medium having instructions stored thereon is disclosed. The instructions, when executed by a computing device, cause the computing device to determine a first temperature associated with a forward power heat source of a microwave energy source with a first temperature sensor, determine a second temperature associated with a termination point of the reverse microwave power with a second temperature sensor, and determine reverse power based on a temperature difference between the first and second temperatures.

In some examples, a microwave energy system is disclosed that includes a microwave energy source, an ablation probe coupled to the microwave energy source to receive a forward power therefrom, a microwave power sensor operably coupled to the microwave energy source, a non-microwave power sensor operably coupled to the microwave energy source, and a control system. The control system is configured to determine, using the microwave power sensor, a first power level associated with the microwave energy source, determine, using the non-microwave power sensor, a second power level associated with the microwave energy source, and determine a state of the microwave power sensor based on the first and second power levels.

In some examples, a method for power detection in an ablation microwave energy system is disclosed that includes determining, using a microwave power sensor, a first power level associated with a microwave energy source, determining, using a non-microwave power sensor, a second power level associated with the microwave energy source, and determining a state of the microwave power sensor based on the first and second power levels.

In some examples, a non-transitory computer readable medium having instructions stored thereon is disclosed. The instructions, when executed by a computing device, cause the computing device to determine, using a microwave power sensor, a first power level associated with a microwave energy source, determine, using a non-microwave power sensor, a second power level associated with the microwave energy source, and determine a state of the microwave power sensor based on the first and second power levels.

In some examples, the microwave power sensor can be a forward microwave power sensor. In these examples, determining the second power level associated with the microwave energy source can include measuring a current of power supplied to the microwave energy source with a current sensor of the plurality of sensors, measuring a voltage of the power supplied to the microwave energy source with a voltmeter of the plurality of sensors, determining the power supplied to the microwave energy source with the current and the voltage, determining lost power due to inefficiency of the microwave energy source, and determining the second power level by subtracting the lost power from the power supplied to the microwave energy source

In some examples, the microwave power sensor can be a reverse microwave power sensor. In these examples, determining the second power level associated with the microwave energy source can include measuring a forward temperature associated with a forward power heat source of the microwave energy source with a first temperature sensor of the plurality of sensors, measuring a reverse temperature associated with a termination point of the reverse microwave power with a second temperature sensor of the plurality of sensors, and determining the second power level based on a temperature difference between the forward temperature and the reverse temperature.

In some examples, a microwave energy system is disclosed that includes a microwave energy source, a reverse microwave power sensor operably coupled to the microwave energy source, and a control system. The control system is configured to control the microwave energy source to output forward power at a plurality of frequencies, monitor reverse power received at the microwave energy source with the reverse microwave power sensor, and analyze the reverse power measured by the reverse microwave power sensor with reference to the plurality of frequencies to determine a state of the reverse power microwave sensor.

In some examples, a method for redundant reverse power detection in a microwave energy system is disclosed that includes controlling a microwave energy source to output forward power at a plurality of frequencies, monitoring reverse power received at the microwave energy source with a reverse microwave power sensor, and analyzing the reverse power measured by the reverse microwave power sensor with reference to the plurality of frequencies to determine a state of the reverse power microwave sensor.

In some examples, a non-transitory computer readable medium having instructions stored thereon is disclosed. The instructions, when executed by a computing device, cause the computing device to control a microwave energy source to output forward power at a plurality of frequencies, monitor reverse power received at the microwave energy source with a reverse microwave power sensor, and analyze the reverse power measured by the reverse microwave power sensor with reference to the plurality of frequencies to determine a state of the reverse power microwave sensor.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a microwave energy system according to some embodiments.

FIG. 2 is a simplified diagram of the microwave energy system of FIG. 1 including sensors according to some embodiments.

FIG. 3 is a simplified diagram of components of a microwave energy source of the microwave energy system of FIG. 1 including sensors according to some embodiments.

FIG. 4 is a sectional perspective view of a microwave energy source according to some embodiments.

FIG. 5 is a flowchart illustrating a method for detecting the state of microwave power sensors in an ablation microwave energy system according to some embodiments.

FIG. 6 is a flowchart illustrating a method for redundant forward power detection in a microwave energy system according to some embodiments.

FIG. 7 is a flowchart illustrating a method for redundant reverse power detection in a microwave energy system according to some embodiments.

FIG. 8 is a flowchart illustrating a method for redundant reverse power detection in a microwave energy system according to some embodiments.

FIG. 9 is a simplified diagram of a medical system according to some embodiments.

FIG. 10A is a simplified diagram of a medical instrument system according to some embodiments.

FIG. 10B is a simplified diagram of a medical instrument including a medical tool within an elongate device according to some embodiments.

FIGS. 11A and 11B are simplified diagrams of side views of a patient coordinate space including a medical instrument mounted on an insertion assembly according to some embodiments.

FIG. 12 is a graph showing differences in temperature measurements from two temperature sensors coupled to a microwave generator at various forward power level settings with a well-matched load.

FIG. 13 is a graph showing the same differences in temperature measurements as FIG. 12 with the addition of test cases having 60% reflection.

FIG. 14 is a graph showing the first 50 seconds of FIG. 13.

FIG. 15 is a graph showing the same differences in temperature measurements as FIG. 12 with the addition of test cases having 100% reflection.

FIG. 16 is a graph showing the first 50 seconds of FIG. 15.

FIG. 17 is a graph showing differences in temperature measurements from two temperature sensors coupled to a microwave generator for a set forward power at various reverse power levels.

FIG. 18 is a graph showing the first 50 seconds of FIG. 17.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Microwave ablation can use a microwave energy source (e.g., a generator) along with the ability to sense the outgoing or forward microwave power and the reflected or reverse microwave power reflected backward into the microwave energy source due to impedance mismatches in the cable or the load (ablation antenna in the patient). In a microwave ablation system, detecting forward power enables a feedback loop for accurately maintaining an output and determining the amount of power that leaves the microwave generator to be delivered to a patient. Detecting reflected power helps the system to determine the amount of power actually delivered to the patient by subtracting the reflected power, which can vary dramatically throughout a procedure, from the delivered power. Accurate power measurements allow the system to ensure that a desired amount of microwave power is delivered to the patient for an ablation procedure. Due to this consideration, redundant measurements can be added to the ablation system to ensure that the system sensors are operating correctly. As compared to microwave power sensors, the systems and methods disclosed herein provide redundant measurements in ways that may be simpler, smaller, and/or less expensive.

In some examples, a redundant forward power measurement can be provided by measuring the input DC power to the microwave energy source and then subtracting the power loss resulting from (e.g., pre-measured) inefficiency of the microwave energy source. In further examples, a temperature of the microwave energy source can be measured during an ablation procedure because inefficiency is temperature dependent for the output stage and measuring the temperature allows the systems and methods to determine power lost to heat. The relationship between inefficiency values and temperature at one or more locations of the microwave energy source can be calibrated, such as before a procedure (e.g., during development, manufacturing, or onsite).

The inefficiency of the microwave energy source may also drift throughout the life of the device, but can be tracked with repeat calibration including self-calibration. Any drift in the inefficiency can also be adjusted for by comparing the temperature adjacent to the output stage to ambient temperature over the life of the device, as discussed in more detail below. Furthermore, storing a temperature history for the microwave energy source over time may provide a better illustration of a trend in changes to the inefficiency of the microwave energy source.

In some examples, a redundant reverse power measurement can be provided by measuring a reverse power temperature caused by the termination of reverse microwave power near a termination point (e.g., isolator resistor) and comparing the reverse power temperature to a forward power temperature near a main heat source of the microwave energy source (e.g., a transistor at the output stage). When reflected power is small, the forward power temperature at the output stage will be a pre-measured number of degrees higher than the reverse power temperature at the termination point. When reflected power is larger, however, the differential temperature measurement partially or entirely inverts because the reverse power is converted into heat at the termination point (e.g., by the isolator's termination resistor). This configuration nominally measures the temperature difference between the hottest point caused by forward power (e.g., inefficiency of the output stage) with respect to the hottest point caused by the reverse power (e.g., termination at the isolator). In some embodiments, the temperature gradient across the entire microwave energy source may be analyzed (e.g., using an infrared camera) both with and without reverse power to find locations with the greatest difference in temperature measurements where a first temperature sensor would be effectively heated by its respective target (e.g. the output stage or termination point) and simultaneously minimally heated by the target of a second temperature sensor (e.g., the output stage or termination point).

In some examples, a frequency sweep (e.g., within a predetermined frequency band) may alternatively or additionally be used as a redundant check on operation of the reverse power measurement sensor. An impedance mismatch anywhere between the load and forward microwave power source creates reflections that reduce power transfer and result in a standing wave. A frequency sweep shifts the location of the peaks and valleys of the standing wave and may change to a frequency with improved or reduced load to source impedance matching and therefore improve or reduce power transfer. As such, a frequency sweep may be used to confirm the functionality of the reverse power sensor of the microwave energy source based on the relationship between frequency and changing reflected power measurement. In one example, if increasing frequency from an original value moves the standing wave at the reverse power sensor away from a peak, then reducing the frequency to the original value should move the standing wave back towards the peak. This process can be performed repeatedly to check on the operation of the reverse power measurement sensor as real-time sensor interrogation.

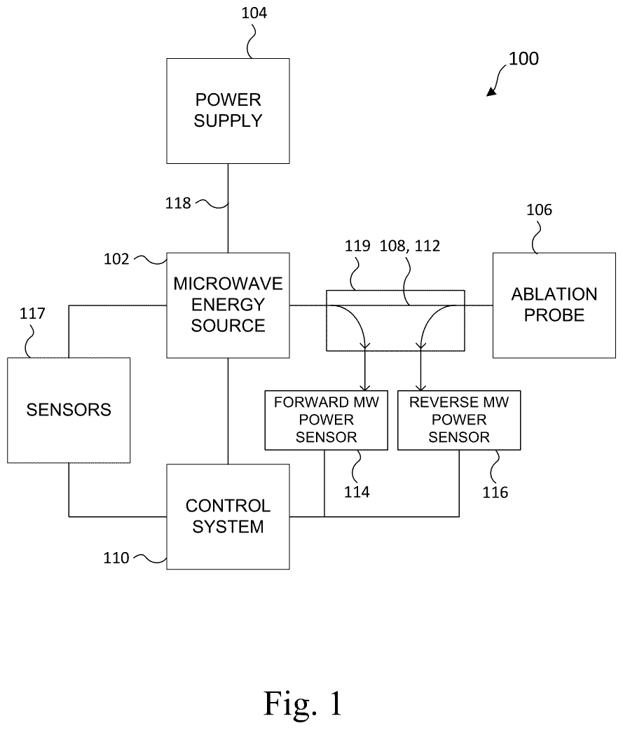

An example microwave energy system 100 is shown diagrammatically in FIGS. 1-3. The system 100 includes a microwave energy source 102 (e.g., a power amplifier) having a power supply 104. An ablation probe 106 is coupled to the microwave energy source 102 to receive a forward power 108 from the microwave energy source 102 for ablation procedures. A control system 110 is operably coupled to the microwave energy source 102 and configured to control the operation of the microwave energy source 102, including monitoring the forward power 108 sent from the microwave energy source 102, as well as reverse power 112 received at the microwave energy source 102 from the ablation prove 106.

As discussed above, the microwave energy system 100 can further include forward microwave power sensor 114 and reverse microwave power sensor 116. The forward microwave power sensor 114 is configured to provide the control system 110 with measurements of the forward power 108 during operation of the microwave energy source 102. The reverse microwave power sensor 116 is configured to provide the control system 110 with measurements of the reverse power 112 during operation of the microwave energy source 102. In one example, as shown in FIG. 1, the forward and reverse microwave power sensors 114, 116 can receive a small fraction (e.g., −40 dB) of the respective power via a directional coupler 119 which directs the fractional power to the respective sensor 114, 116.

To ensure that the forward and reverse microwave power sensors 114, 116 are operating correctly and power levels can be accurately tracked while using the microwave energy system 100, the system 100 can further include redundant measurements of the forward power 108 sent from the microwave energy source 102 and/or the reverse power 112 received at the microwave energy source 102.

Pursuant to this, in some examples, the system 100 can include a plurality of sensors 117 that provide data sufficient for the control system 110 to determine forward power and/or reverse power independent of the forward and/or reverse microwave power sensors 114, 116. The independent determination allows the control system 110 to compare the measured values and determine a state (e.g., operational, error, etc.) of the forward and/or reverse microwave power sensors 114, 116.

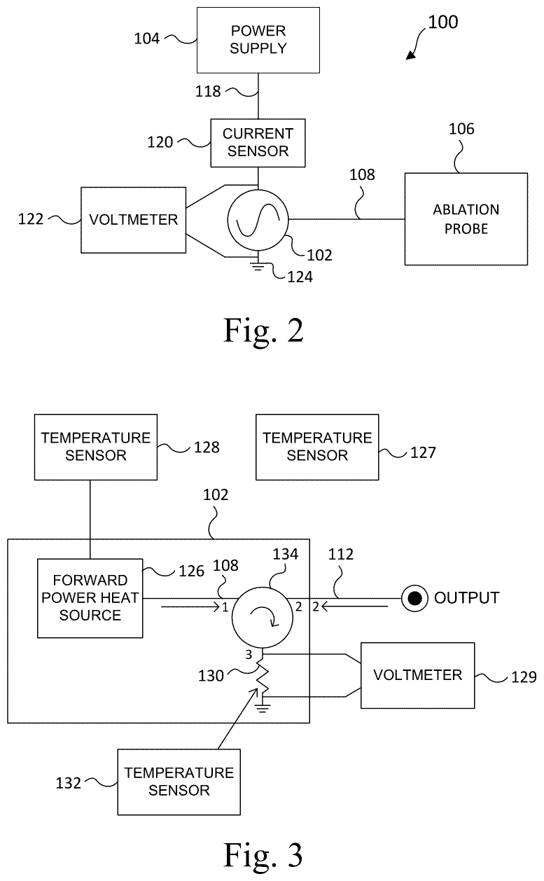

In one example, control system 110 can determine the forward power 108 based on the power supplied 118 to the microwave energy source 102 and an inefficiency of the microwave energy source 102. Pursuant to this, the sensors 117 can be utilized to allow the control system 110 to measure the power supplied 118 to the microwave energy source 102 by the power supply 104. As shown in FIG. 2, the sensors 117 can include a current sensor 120 and a voltmeter 122. The current sensor 120 is configured to measure a current of the power supplied 118 to the microwave energy source 102 by the power supply 104. The voltmeter 122 is coupled across the microwave energy source 102 (including to ground 124 at one terminal) to measure a supply voltage from the power supply 104. The value of the power supplied 118 to microwave energy source 102 can then be determined by the control system 110 as needed by multiplying the current and supply voltage.

The control system 110 can determine lost power due to an inefficiency of the microwave power source 102. Using the power supplied 118 to the microwave energy source 102 and the inefficiency of the microwave power source 102, the control system 110 can determine the forward power 108 provided by the microwave energy source 102 to the ablation probe 106 by subtracting the inefficiency of the microwave energy source 102 from the power supplied 118 by the power supply 104. In some examples, the control system 110 may adjust the microwave energy source 102 based on the forward power 108, as determined by the forward microwave power sensor 114 and/or the redundant measurements.

In some embodiments, the inefficiency of the microwave energy source 102 can be correlated to a temperature measurement of a forward power heat source 126 (e.g., the output stage) of the microwave energy source 102 associated with the forward power 108, such as an output stage of the microwave energy source 102. Utilizing this pre-determined relationship, the relationship between inefficiency values of the microwave energy source 102 and the temperature at the heat source 126 can be calibrated, such that the control system 110 can determine an inefficiency of the microwave energy source 102 based on monitoring temperature of the forward power heat source 126 of the microwave energy source 102. The control system 110 uses the determined inefficiency of the microwave energy source 102 to determine the redundant measurement of the forward power 108.

As such, the plurality of sensors 117 can include a temperature sensor 128 disposed at a location suitable to measure the temperature of the forward power heat source 126, such as at, near, or adjacent to the forward power heat source 126. With this configuration, the control system 110 can monitor the temperature of the forward power heat source 126 to determine whether the inefficiency of the microwave energy source 102 has changed because an inefficiency increase will cause greater heating at the forward power heat source 126. If desired, the control system 110 can be configured to monitor changes in the inefficiency of the microwave energy source 102 over time to update the predetermined correlation between temperature measured by the temperature sensor 128 and the inefficiency.

In some examples, the control system 110 can be configured to track the temperature of the forward power heat source 126 with respect to ambient temperature over time. The ambient temperature measurement ensures the accuracy of the measurements over time because different ambient temperatures can affect the temperature of the forward power heat source 126 independent of inefficiencies of the microwave energy source 102. For example, if the forward power heat source 126 becomes hotter over time for the same forward power 108 and ambient temperature, then the microwave energy source 102 has increased inefficiency. As such, the measured temperature of the forward power heat source 126 may be adjusted using the ambient temperature and then used to determine the inefficiency of the microwave energy source 102. In some embodiments, as shown in FIG. 3, a temperature sensor 127 located away from the forward power heat source 126 (and any other non-ambient heat sources) may be used to measure the ambient temperature. Upon detection of the inefficiency drift, the control system 110 may adjust the predetermined inefficiency value of the microwave energy source 102 used to determine the forward power 108. For example, to make such an adjustment as accurate as possible, the forward power heat source 126 can be monitored in a system configuration without reverse power or by subtracting heating caused by reverse power, as discussed in more detail below.

In another example the control system 110 can determine a redundant measure of the reverse power 112 with a voltmeter 129 that measures voltage across a reverse power heat source 130 associated with the reverse power 112, which in this example is a terminating resistor of an isolator having a circulator, as discussed in more detail below. In this example, the reverse power 112 is determined by the voltage across the reverse power heat source 130 squared, divided by the resistance of the terminating resistor.

In another example, the control system 110 can determine the redundant measure of the reverse power 112 based on a temperature difference between the forward power heat source 126 associated with the forward power 108 and a reverse power heat source 130 associated with the reverse power 112. Accordingly, as shown in FIG. 3, the plurality of sensors 117 can include the temperature sensor 128 disposed adjacent to the forward power heat source 126 as a first temperature sensor and a second temperature sensor 132 disposed adjacent to the reverse power heat source 130. Utilizing the first and second temperature sensors 128, 132, the control system 110 can determine the reverse power 112 based on a temperature difference between the temperatures measured by the first and second temperature sensors 128, 132.

Examples of the forward power heat source 126 and the reverse power heat source 130 are shown in FIG. 3. As shown, the forward power heat source 126 can be an output stage of the microwave energy source 102 and/or the reverse power heat source 130 can be a termination point of the microwave energy source 102. In further examples, the output stage can be an output stage transistor of the microwave energy source 102 and/or the termination point can be an isolator resistor.

In one form, as shown in FIG. 3, the microwave energy source 102 includes a circulator 134 to control power flow from and to the microwave energy source 102. The circulator 134 is a passive, non-reciprocal three-port device that directs microwave power input into one of the ports to exit through a next adjacent port. For example, microwave power can be directed to a next adjacent port in a clockwise manner as shown or a counterclockwise manner. As such, the forward power 108 enters in port 1 and exits port 2 to proceed to the ablation probe 106. Likewise, the reverse power 112 enters port 2 and exits port 3 to proceed to the reverse power heat source 130.

Advantageously, pursuant to the above, the microwave energy source 102 can include the forward power heat source 126 (e.g., the output stage), the reverse power heat source 130 (e.g., the isolator resistor), and the circulator 134, while separate components can be utilized to provide redundant sensing as described herein. The separate components can include the temperature sensor 128 associated with the forward power heat source 126, the temperature sensor 132 associated with the reverse power heat source 130, and the voltmeter 129 associated with the reverse power heat source 130. Of course, if desired, one or more of the temperature sensors 128, 132 and voltmeter 129 can be incorporated into the microwave energy source 102 either during initial assembly or as a retrofit component.

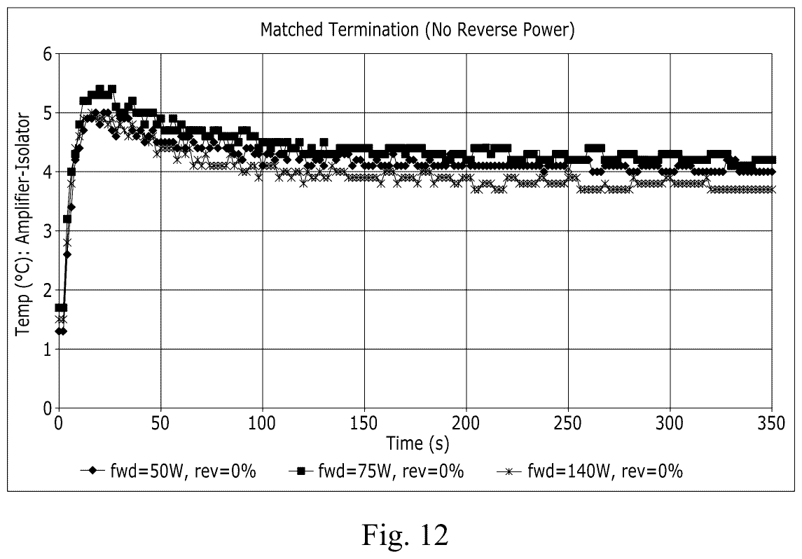

When reverse power is small (e.g., reverse power is 20W or less when forward power is 140 W, about 20% or less of forward power, or about 15% or less of forward power; see also data shown in FIGS. 12-18), the temperature at the forward power heat source 126 (e.g., output stage) will be (e.g., a number of degrees) higher than at the reverse power heat source 130 (e.g., termination resistor). When reverse power is larger, however, the differential temperature measurement may decrease or invert due to the reverse power being converted to heat by the termination resistor.

Upon detecting a larger reverse power by identifying a decrease or inversion of the differential temperature measurement or with analysis of the trendline of the differential measurement (i.e., shape of the differential measurement curve), the control system 110 can check a measurement of the reverse microwave power sensor 116 to make sure that the reverse microwave power sensor 116 is measuring a reverse power, or otherwise is in accordance with the redundant reverse power measurement. In situations where the control system 110 identifies the decrease or inversion of the differential temperature measurement between the sensors 128, 132, but does not receive a reverse power measurement from the reverse microwave power sensor 116 or the reverse power from the reverse microwave power sensor 116 is inconsistent with the redundant reverse power measurement, this may indicate the reverse microwave power sensor 116 is not operational or faulty.

According to one example, the control system 110 may store a relationship between reverse power and the difference between the first and second temperatures measured by the sensors 128, 132. With this configuration, the control system 110 can compare measured values of the first and second temperature to pre-measured values of the microwave energy source 102 under possible use conditions to detect reverse power.

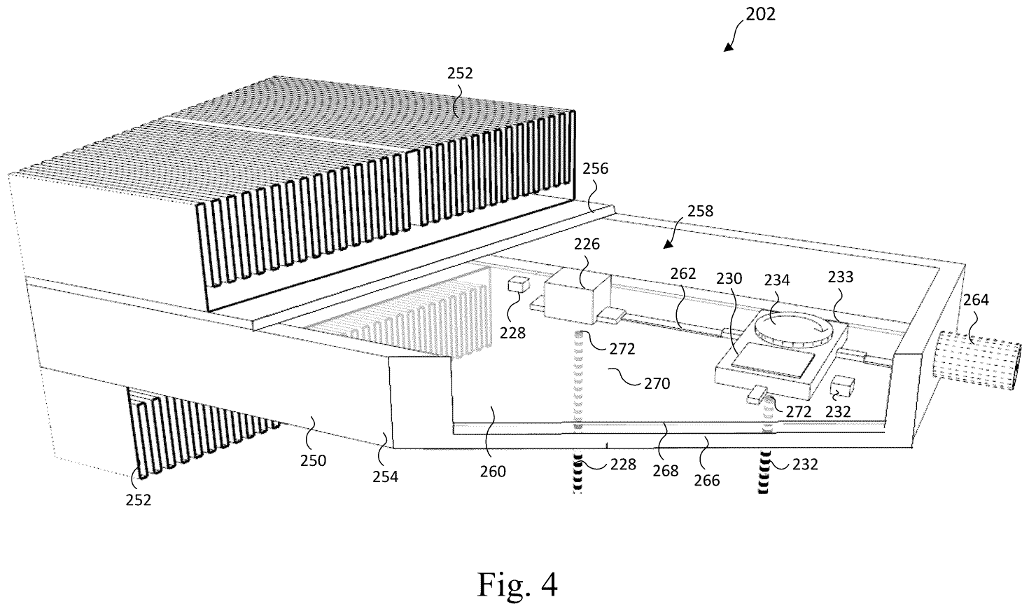

An example microwave energy source 202 is shown in FIG. 4. In some embodiments, the microwave energy source 202 of this embodiment may correspond to the microwave energy source 102 described above. As such, for case of understanding, similar components are referenced with similar reference characters.

As shown, the microwave energy source 202 includes a housing 250 and a heat sink 252 disposed on one or both sides of the housing 250. The housing 250 includes a base 254 and cover 256 defining an interior 258 to receive components therein. Within the interior 258, the microwave energy source 202 includes a circuit board 260 (e.g., a printed circuit board) having a forward power heat source 226 in the form of an output stage transistor and a reverse power heat source 230 in the form of a termination resistor. An isolator 233 including the reverse power heat source 230 and a circulator 234 is disposed along a power line 262 extending from the output stage transistor 226 to a microwave output connector 264 to couple the microwave energy source 202 to an ablation probe (not shown). As discussed above, the circulator 234 functions to direct reverse power to the termination resistor 230. The microwave energy source 202 further includes temperature sensors 228, 232 configured to measure a temperature associated with the output stage transistor 226 and a temperature associated with the termination resistor 230, respectively.

In a first form, the temperature sensors 228, 232 are mounted to the circuit board 260. As such, the first temperature sensor 228 is mounted to the circuit board adjacent to the output stage transistor 226 and the second temperature sensor 232 is mounted to the circuit board adjacent to the termination resistor 230.

If desired, the temperature sensors 228, 232 can be located on the circuit board 260 to minimize a thermal impact from the other sensor's target. As such, the first temperature sensor 228 can be disposed on a side of the output stage transistor 226 furthest from the termination resistor 230 and the second temperature sensor 232 can be disposed on a side of the termination resistor 230 furthest from the output stage transistor 226. In other words, both temperature sensors 228, 232 are as close to their respective heat sources 226, 230, while being furthest from the other's heat source 226, 230.

To optimize the location of the temperature sensors 228, 232, a temperature gradient of the microwave energy source 202, such as that captured by a thermal imaging camera or the like, can be analyzed to find the locations of minimal thermal impact of the other heat sources 226, 230. For example, the temperature gradient can be analyzed in configurations with and without reverse power.

In a second form, the temperature sensors 228, 232 can be external temperature sensors that penetrate through the housing 250 and, optionally, one of the heatsinks 252 to be disposed adjacent to the heat sources 226, 230. In further examples, the sensors 228, 232 can be disposed on an opposite side of the circuit board 260 as the heat sources 226, 230 and, if desired, may also penetrate the circuit board 260 to directly contact the output stage transistor 226 and termination resistor 230, respectively.

In the illustrated form, the circuit board 260 extends along a bottom wall 266 of the housing base 254 such that a bottom surface 268 of the circuit board 260 abuts the bottom wall 266. The microwave power components, including the output stage transistor 226, the termination resistor 230, the circulator 234, and the power line 262 are mounted to an upper surface 270 of the circuit board 260. As shown, the temperature sensors 228, 232 of this form penetrate through the bottom heatsink 252 and the bottom wall 266 of the housing base 254 to be disposed adjacent to or contacting the bottom surface 268 of the circuit board 260 aligned with the respective heat sources 226, 230. In some instances, to further enhance the measurements, the temperature sensors 228, 232 may contact a thermally efficient interface 272 (e.g., thermal paste) disposed on the bottom surface 268 of the circuit board 260.

In another example, the control system 110 can confirm that the reverse microwave power sensor 116 is operational by analyzing measurements of the sensor 116 when controlling the microwave energy source 102 at a plurality of frequencies. In this example, the control system 110 controls the microwave energy source 102 to output the forward power 108 at a plurality of frequencies.

An impedance mismatch in the cable or the load creates reflections that reduce power transfer and may result in a standing voltage wave. Changing the frequencies of the microwave energy source 102 can improve matching and, therefore, improve power transfer. Different frequencies also shift the location of the peaks and valleys of the standing wave with respect to the reverse power microwave sensor 116, which may affect its measurement.

Utilizing this relationship, the control system 110 can monitor the reverse power 112 received at the microwave energy source 102 with the reverse microwave power sensor 116 while the microwave energy source 102 outputs the forward power 108 at the plurality of frequencies. The control system 110 then analyzes the reverse power 112 measured by the reverse microwave power sensor 116 with reference to the plurality of frequencies to determine a state (e.g., operational, error, etc.) of the reverse power microwave sensor 116. For example, the control system 110 can analyze the value at different points of the standing wave while shifting between the plurality of frequencies with reference to the reverse power 112 measured by the reverse microwave power sensor 116.

In some examples, the control system 110 may monitor the causal relationship between the frequency changes and corresponding changes in reflected power measurement by the reverse power microwave sensor 116 to determine the state of the reverse power microwave sensor 116.

In some examples, the plurality of frequencies can be within a predetermined range and/or a sweep of the plurality of frequencies can be performed sequentially/repeatedly. One example predetermined range can be a predetermined band corresponding to a particular operation, such as a 2.4 GHz-2.5 GHz band or a 902 MHZ-928 MHz band, pursuant to an ablation procedure.

Although the above description includes references to an ablation procedure and a corresponding ablation probe, it will be understood that the systems and methods described herein can be applicable to other suitable procedures utilizing an energy source, such as electroporation.

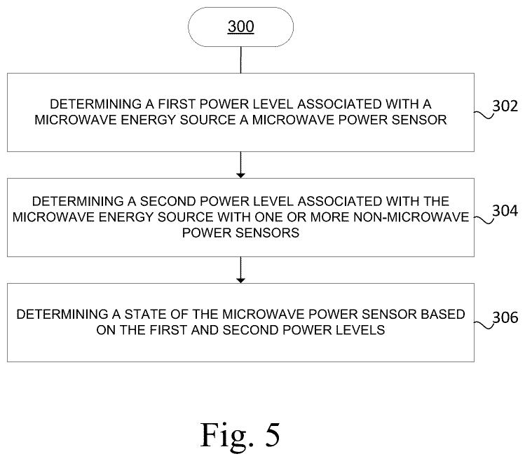

FIG. 5 illustrates a method 300 for power detection in an ablation microwave energy system (e.g., system 100). The method 300 is illustrated as a set of operations or processes 302 through 306. Not all of the illustrated processes may be performed in all embodiments of method 300. Additionally, one or more processes that are not expressly illustrated in FIG. 5 may be included before, after, in between, or as part of the processes 302 through 306. Processes may also be performed in different orders. In some embodiments, one or more of the processes 302 through 306 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 302 through 306 may be performed by a controller (e.g., control system 110).

In process 302, a microwave power sensor (e.g., microwave power sensor 114, 116) determines a first power level associated with a microwave energy source (e.g., microwave energy source 102). In process 304, a control system (e.g., control system 110) determines a second power level associated with the microwave energy source with one or more non-microwave power sensors (e.g., sensors 117, 120, 122, 128, 132). In process 306, the control system determines a state of the microwave power sensor based on the first and second power levels.

In one example, process 304 includes measuring a current of power supplied (e.g., power supplied 118) to the microwave energy source with a current sensor (e.g., current sensor 120), measuring a voltage of the power supplied to the microwave energy source with a voltmeter (e.g., voltmeter 122), determining the power supplied to the microwave energy source with the current and the voltage, determining lost power due to inefficiency of the microwave energy source, and determining the second power level by subtracting the lost power from the power supplied to the microwave energy source. In this example, the microwave power sensor can be a forward microwave power sensor (e.g., forward microwave power sensor 114). This example can also include an additional process of measuring a temperature associated with the microwave energy source (e.g., forward power heat source 126) with a temperature sensor (e.g., temperature sensor 128), wherein the inefficiency of the microwave energy source is at least partially determined based on the temperature.

In another example, process 304 includes measuring a forward temperature associated with a forward power heat source (e.g., forward power heat source 126) of the microwave energy source with a first temperature sensor (e.g., temperature sensor 128), measuring a reverse temperature associated with a termination point (e.g., reverse power heat source 130) of the microwave energy source with a second temperature sensor (e.g., temperature sensor 132), and determining the second power level based on a temperature difference between the forward temperature and the reverse temperature. In this example, the microwave power sensor can be a reverse microwave power sensor (e.g., reverse microwave power sensor 116).

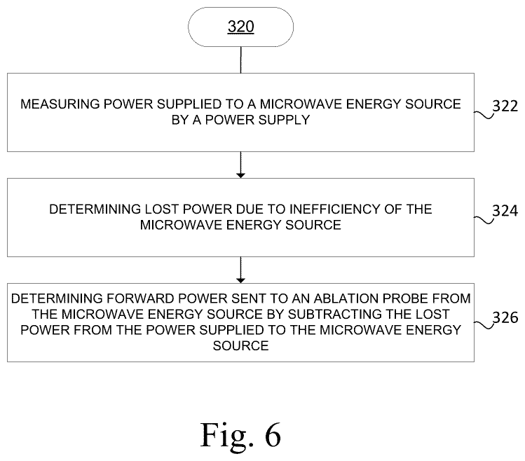

FIG. 6 illustrates a method 320 for redundant forward power detection in a microwave energy system (e.g., system 100). The method 320 is illustrated as a set of operations or processes 322 through 326. Not all of the illustrated processes may be performed in all embodiments of method 320. Additionally, one or more processes that are not expressly illustrated in FIG. 6 may be included before, after, in between, or as part of the processes 322 through 326. Processes may also be performed in different orders. In some embodiments, one or more of the processes 322 through 326 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 322 through 326 may be performed by a controller (e.g., control system 110).

At process 322, power supplied (e.g., power supplied 118) to a microwave energy source (e.g., microwave energy source 102) by a power supply (e.g., power supply 104) is measured. At process 324, lower power due to inefficiency of the microwave energy source is determined. At process 326, forward power (e.g., forward power 108) sent to an ablation probe (e.g., ablation probe 106) from the microwave energy source is determined by subtracting the lost power from the power supplied to the microwave energy source.

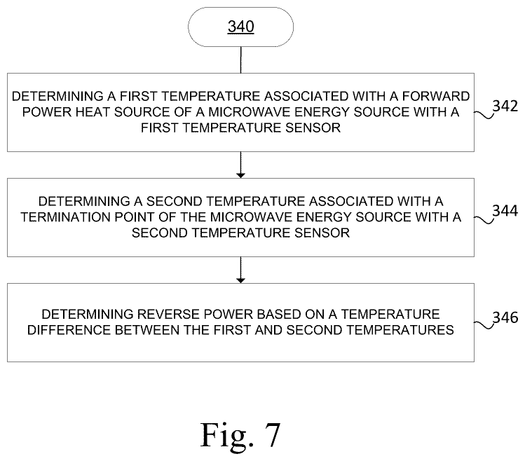

FIG. 7 illustrates a method 340 for redundant reverse power detection in a microwave energy system (e.g., system 100). The method 340 is illustrated as a set of operations or processes 342 through 346. Not all of the illustrated processes may be performed in all embodiments of method 340. Additionally, one or more processes that are not expressly illustrated in FIG. 7 may be included before, after, in between, or as part of the processes 342 through 346. Processes may also be performed in different orders. In some embodiments, one or more of the processes 342 through 346 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 342 through 346 may be performed by a controller (e.g., control system 110).

At process 342, a first temperature associated with a forward power heat source (e.g., forward power heat source 126) of a microwave energy source (e.g., microwave energy source 102) is determined with a first temperature sensor (e.g., temperature sensor 128). At process 344, a second temperature associated with a termination point (e.g., reverse power heat source 130) of the microwave energy source with a second temperature sensor (e.g., temperature sensor 132). At process 346, reverse power is determined based on a temperature difference between the first and second temperatures.

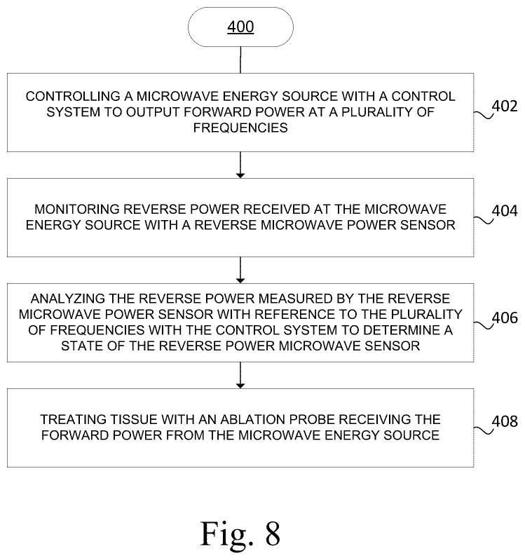

FIG. 8 illustrates a method 400 for redundant reverse power detection in a microwave energy system (e.g., system 100). The method 400 is illustrated as a set of operations or processes 402 through 408. Not all of the illustrated processes may be performed in all embodiments of method 400. Additionally, one or more processes that are not expressly illustrated in FIG. 8 may be included before, after, in between, or as part of the processes 402 through 408. Processes may also be performed in different orders. In some embodiments, one or more of the processes 402 through 408 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 402 through 408 may be performed by a controller (e.g., control system 110).

At process 402, a microwave energy source (e.g., microwave energy source 102) is controlled by a control system (e.g., control system 110) to output forward power (e.g., forward power 108) at a plurality of frequencies. At process 404, a reverse microwave power sensor (e.g., reverse microwave power sensor 116) monitors reverse power (e.g., reverse power 112) received at the microwave energy source. At process 406, the control system analyzes the reverse power measured by the reverse microwave power sensor with reference to the plurality of frequencies to determine a state of the reverse power microwave sensor. At process 408, tissue is treated with an ablation probe (e.g., ablation probe 106) receiving the forward power from the microwave energy source.

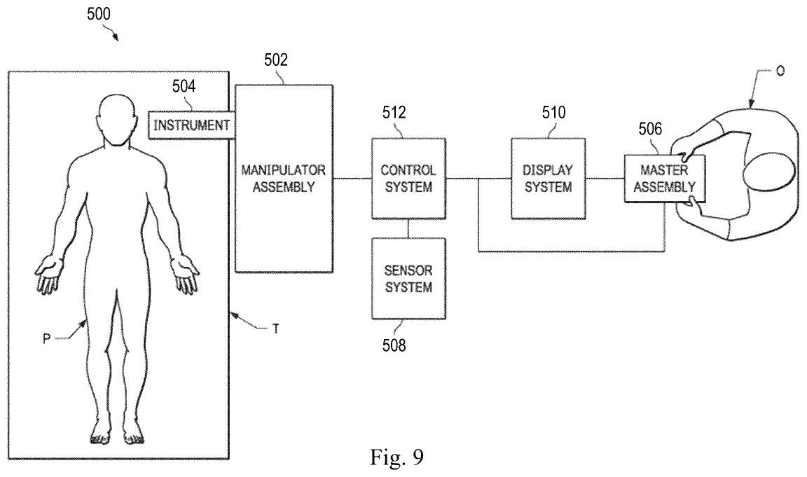

In various embodiments, any of the described energy delivery systems may be used as a medical instrument delivered by, coupled to, and/or controlled by a teleoperated medical system. FIG. 9 is a simplified diagram of a teleoperated medical system 500 according to some embodiments. In some embodiments, teleoperated medical system 500 may be suitable for use in, for example, surgical, diagnostic, therapeutic, or biopsy procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic or teleoperational systems.

FIG. 9 is a simplified diagram of a medical system 500 according to some embodiments. The medical system 500 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 9, medical system 500 may include a manipulator assembly 502 that controls the operation of a medical instrument 504 in performing various procedures on a patient P. Medical instrument 504 may extend into an internal site within the body of patient P via an opening in the body of patient P. The manipulator assembly 502 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 502 may be mounted to and/or positioned near a patient table T. A master assembly 506 allows an operator O (e.g., a surgeon, a clinician, a physician, or other user) to control the manipulator assembly 502. In some examples, the master assembly 506 allows the operator O to view the procedural site or other graphical or informational displays. In some examples, the manipulator assembly 502 may be excluded from the medical system 500 and the instrument 504 may be controlled directly by the operator O. In some examples, the manipulator assembly 502 may be manually controlled by the operator O. Direct operator control may include various handles and operator interfaces for hand-held operation of the instrument 504.

The master assembly 506 may be located at a surgeon's console which is in proximity to (e.g., in the same room as) a patient table T on which patient P is located, such as at the side of the patient table T. In some examples, the master assembly 506 is remote from the patient table T, such as in in a different room or a different building from the patient table T. The master assembly 506 may include one or more control devices for controlling the manipulator assembly 502. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 502 supports the medical instrument 504 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 512). The manipulator assembly 502 may include a plurality of actuators (e.g., motors) that drive inputs on the medical instrument 504 in response to commands, such as from the control system 512. The actuators may include drive systems that move the medical instrument 504 in various ways when coupled to the medical instrument 504. For example, one or more actuators may advance medical instrument 504 into a naturally or surgically created anatomic orifice. Actuators may control articulation of the medical instrument 504, such as by moving the distal end (or any other portion) of medical instrument 504 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 504, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 504.

The medical system 500 may include a sensor system 508 with one or more sub-systems for receiving information about the manipulator assembly 502 and/or the medical instrument 504. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 504; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 504 or from some other location; and/or actuator position sensors such as resolvers, encoders, potentiometers, and the like that describe the rotation and/or orientation of the actuators controlling the medical instrument 504.

The medical system 500 may include a display system 510 for displaying an image or representation of the procedural site and the medical instrument 504. Display system 510 and master assembly 506 may be oriented so physician O can control medical instrument 504 and master assembly 506 with the perception of telepresence.

In some embodiments, the medical instrument 504 may include a visualization system, which may include an image capture assembly that records a concurrent or real-time image of a procedural site and provides the image to the operator O through one or more displays of display system 510. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 504. Additionally or alternatively, a separate endoscope, attached to a separate manipulator assembly, may be used with medical instrument 504 to image the procedural site. The visualization system may be implemented as hardware, firmware, software or a combination thereof which interact with or are otherwise executed by one or more computer processors, such as of the control system 512.

Display system 510 may also display an image of the procedural site and medical instruments, which may be captured by the visualization system. In some examples, the medical system 500 provides a perception of telepresence to the operator O. For example, images captured by an imaging device at a distal portion of the medical instrument 504 may be presented by the display system 510 to provide the perception of being at the distal portion of the medical instrument 504 to the operator O. The input to the master assembly 506 provided by the operator O may move the distal portion of the medical instrument 504 in a manner that corresponds with the nature of the input (e.g., distal tip turns right when a trackball is rolled to the right) and results in corresponding change to the perspective of the images captured by the imaging device at the distal portion of the medical instrument 504. As such, the perception of telepresence for the operator O is maintained as the medical instrument 504 is moved using the master assembly 506. The operator O can manipulate the medical instrument 504 and hand controls of the master assembly 506 as if viewing the workspace in substantially true presence, simulating the experience of an operator that is physically manipulating the medical instrument 504 from within the patient anatomy.

In some examples, the display system 510 may present virtual images of a procedural site that are created using image data recorded pre-operatively (e.g., prior to the procedure performed by the medical instrument system 600) or intra-operatively (e.g., concurrent with the procedure performed by the medical instrument system 600), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 510 may display a virtual image that is generated based on tracking the location of medical instrument 504. For example, the tracked location of the medical instrument 504 may be registered (e.g., dynamically referenced) with the model generated using the pre-operative or intra-operative images, with different portions of the model correspond with different locations of the patient anatomy. As the medical instrument 504 moves through the patient anatomy, the registration is used to determine portions of the model corresponding with the location and/or perspective of the medical instrument 504 and virtual images are generated using the determined portions of the model. This may be done to present the operator O with virtual images of the internal procedural site from viewpoints of medical instrument 504 that correspond with the tracked locations of the medical instrument 504.

The medical system 500 may also include the control system 512, which may include processing circuitry that implements the some or all of the methods or functionality discussed herein. The control system 512 may include at least one memory and at least one processor for controlling the operations of the manipulator assembly 502, the medical instrument 504, the master assembly 506, the sensor system 508, and/or the display system 510. Control system 512 may include instructions (e.g., a non-transitory machine-readable medium storing the instructions) that when executed by the at least one processor, configures the one or more processors to implement some or all of the methods or functionality discussed herein. While the control system 512 is shown as a single block in FIG. 9, the control system 512 may include two or more separate data processing circuits with one portion of the processing being performed at the manipulator assembly 502, another portion of the processing being performed at the master assembly 506, and/or the like. In some examples, the control system 512 may include other types of processing circuitry, such as application-specific integrated circuits (ASICs) and/or field-programmable gate array (FPGAs). The control system 512 may be implemented using hardware, firmware, software, or a combination thereof.

In some examples, the control system 512 may receive feedback from the medical instrument 504, such as force and/or torque feedback. Responsive to the feedback, the control system 512 may transmit signals to the master assembly 506. In some examples, the control system 512 may transmit signals instructing one or more actuators of the manipulator assembly 502 to move the medical instrument 504. In some examples, the control system 512 may transmit informational displays regarding the feedback to the display system 510 for presentation or perform other types of actions based on the feedback.

The control system 512 may include a virtual visualization system to provide navigation assistance to operator O when controlling the medical instrument 504 during an image-guided medical procedure. Virtual navigation using the virtual visualization system may be based upon an acquired pre-operative or intra-operative dataset of anatomic passageways of the patient P. The control system 512 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 508 that is used to compute an (e.g., approximate) location of the medical instrument 504 with respect to the anatomy of patient P. The sensor system 508 may be used to register and display the medical instrument 504 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 508 may be used to compute the (e.g., approximate) location of the medical instrument 504 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 500 may further include operations and support systems (not shown) such as illumination systems, steering control systems, irrigation systems, and/or suction systems. In some embodiments, the medical system 500 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

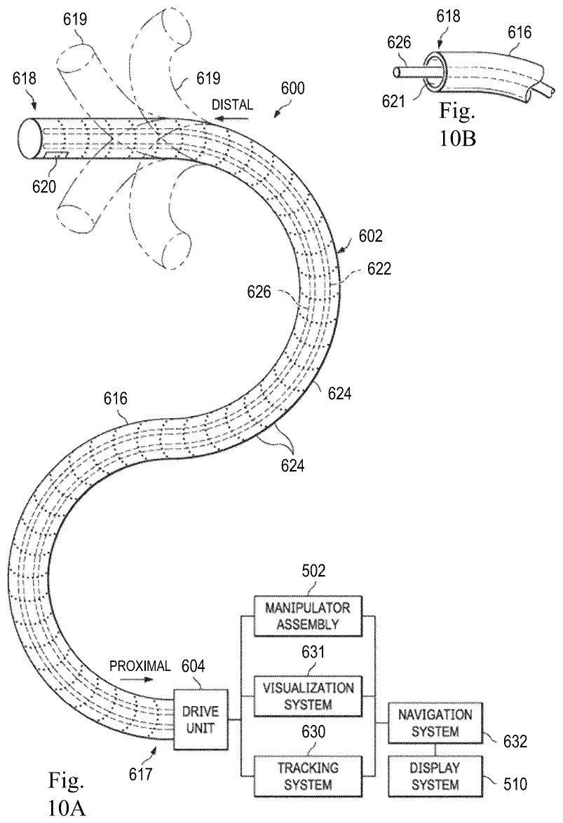

FIG. 10A is a simplified diagram of a medical instrument system 600 according to some embodiments. The medical instrument system 600 includes a flexible elongate device 602 (also referred to as elongate device 602), a drive unit 604, and a medical tool 626 that collectively is an example of a medical instrument 504 of a medical system 500. The medical system 500 may be a teleoperated system, a non-teleoperated system, or a hybrid teleoperated and non-teleoperated system, as explained with reference to FIG. 9. A visualization system 631, tracking system 630, and navigation system 632 are also shown in FIG. 10A and are example components of the control system 512 of the medical system 500. In some examples, the medical instrument system 600 may be used for non-teleoperational exploratory procedures or in procedures involving traditional manually operated medical instruments, such as endoscopy. The medical instrument system 600 may be used to gather (e.g., measure) a set of data points corresponding to locations within anatomic passageways of a patient, such as patient P.

The elongate device 602 is coupled to the drive unit 604. The elongate device 602 includes a channel 621 through which the medical tool 626 may be inserted. The elongate device 602 navigates within patient anatomy to deliver the medical tool 626 to a procedural site. The elongate device 602 includes a flexible body 616 having a proximal end 617 and a distal end 618. In some examples, the flexible body 616 may have an approximately 3 mm outer diameter. Other flexible body outer diameters may be larger or smaller.

Medical instrument system 600 may include the tracking system 630 for determining the position, orientation, speed, velocity, pose, and/or shape of the flexible body 616 at the distal end 618 and/or of one or more segments 624 along flexible body 616, as will be described in further detail below. The tracking system 630 may include one or more sensors and/or imaging devices. The flexible body 616, such as the length between the distal end 618 and the proximal end 617, may include multiple segments 624. The tracking system 630 may be implemented using hardware, firmware, software, or a combination thereof. In some examples, the tracking system 630 is part of control system 512 shown in FIG. 9.

Tracking system 630 may track the distal end 618 and/or one or more of the segments 624 of the flexible body 616 using a shape sensor 622. The shape sensor 622 may include an optical fiber aligned with the flexible body 616 (e.g., provided within an interior channel of the flexibly body 616 or mounted externally along the flexible body 616). In some examples, the optical fiber may have a diameter of approximately 200 μm. In other examples, the diameter may be larger or smaller. The optical fiber of the shape sensor 622 may form a fiber optic bend sensor for determining the shape of flexible body 616. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 616 may be determined using other techniques. For example, a history of the position and/or pose of the distal end 618 of the flexible body 616 can be used to reconstruct the shape of flexible body 616 over an interval of time (e.g., as the flexible body 616 is advanced or retracted within a patient anatomy). In some examples, the tracking system 630 may alternatively and/or additionally track the distal end 618 of the flexible body 616 using a position sensor system 620. Position sensor system 620 may be a component of an EM sensor system with the position sensor system 620 including one or more position sensors. Although the position sensor system 620 is shown as being near the distal end 618 of the flexible body 616 to track the distal end 618, the number and location of the position sensors of the position sensor system 620 may vary to track different regions along the flexible body 616. In one example, the position sensors include conductive coils that may be subjected to an externally generated electromagnetic field. Each coil of position sensor system 620 may produce an induced electrical signal having characteristics that depend on the position and orientation of the coil relative to the externally generated electromagnetic field. The position sensor system 620 may measure one or more position coordinates and/or one or more orientation angles associated with one or more portions of flexible body 616. In some examples, the position sensor system 620 may be configured and positioned to measure six degrees of freedom, e.g., three position coordinates X, Y, Z and three orientation angles indicating pitch, yaw, and roll of a base point. In some examples, the position sensor system 620 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 630 may alternately and/or additionally rely on a collection of pose, position, and/or orientation data stored for a point of an elongate device 602 and/or medical tool 626 captured during one or more cycles of alternating motion, such as breathing. This stored data may be used to develop shape information about the flexible body 616. In some examples, a series of position sensors (not shown), such as EM sensors like the sensors in position sensor 620 or some other type of position sensors may be positioned along the flexible body 616 and used for shape sensing. In some examples, a history of data from one or more of these position sensors taken during a procedure may be used to represent the shape of elongate device 602, particularly if an anatomic passageway is generally static.

FIG. 10B is a simplified diagram of the medical tool 626 within the elongate device 602 according to some embodiments. The flexible body 616 of the elongate device 602 may include the channel 621 sized and shaped to receive the medical tool 626. In some embodiments, the medical tool 626 may be used for procedures such as diagnostics, imaging, surgery, biopsy, ablation, illumination, irrigation, suction, electroporation, etc. Medical tool 626 can be deployed through channel 621 of flexible body 616 and operated at a procedural site within the anatomy. Medical tool 626 may be, for example, an image capture probe, a biopsy tool (e.g., a needle, grasper, brush, etc.), an ablation probe (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 626 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 626 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 621 when the biopsy tool is within the channel 621. The medical tool 626 may be an image capture probe that includes a distal portion with a stereoscopic or monoscopic camera that may be placed at or near the distal end 618 of flexible body 616 for capturing images (e.g., still or video images). The captured images may be processed by the visualization system 631 for display and/or provided to the tracking system 630 to support tracking of the distal end 618 of the flexible body 616 and/or one or more of the segments 624 of the flexible body 616. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 631. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 616 of the elongate device 602 to facilitate visual navigation of the elongate device 602 to a procedural site and then is replaced within the flexible body 616 with another type of medical tool 626 that performs the procedure. In some examples, the image capture probe may be within the flexible body 616 of the elongate device 602 along with another type of medical tool 626 to facilitate simultaneous image capture and tissue intervention, such as within the same channel 621 or in separate channels. A medical tool 626 may be advanced from the opening of the channel 621 to perform the procedure (or some other functionality) and then retracted back into the channel 621 when the procedure is complete. The medical tool 626 may be removed from the proximal end 617 of the flexible body 616 or from another optional instrument port (not shown) along flexible body 616.

In some examples, the elongate device 602 may include integrated imaging capability rather than utilize a removable image capture probe. For example, the imaging device (or fiber- optic bundle) and the light emitters may be located at the distal end 618 of the elongate device 602. The flexible body 616 may include one or more dedicated channels that carry the cable(s) and/or optical fiber(s) between the distal end 618 and the visualization system 631. Here, the medical instrument system 600 can perform simultaneous imaging and tool operations.

In some examples, the medical tool 626 is capable of controllable articulation. The medical tool 626 may house cables (which may also be referred to as pull wires), linkages, or other actuation controls (not shown) that extend between its proximal and distal ends to controllably bend the distal end of medical tool 626, such as discussed herein for the flexible elongate device 602. The medical tool 626 may be coupled to a drive unit 604 and the manipulator assembly 502. In these examples, the elongate device 602 may be excluded from the medical instrument system 600 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sep. 30, 2008 and titled “Passive Preload and Capstan Drive for Surgical Instruments”), which are incorporated by reference herein in their entireties.

The flexible body 616 of the elongate device 602 may also or alternatively house cables, linkages, or other steering controls (not shown) that extend between the drive unit 604 and the distal end 618 to controllably bend the distal end 618 as shown, for example, by broken dashed line depictions 619 of the distal end 618 in FIG. 10A. In some examples, at least four cables are used to provide independent up-down steering to control a pitch of the distal end 618 and left-right steering to control a yaw of the distal end 281. In these examples, the flexible elongate device 602 may be a steerable catheter. Examples of steerable catheters, applicable in some embodiments, are described in detail in PCT Publication WO 2019/018736 (published Jan. 24, 2019 and titled “Flexible Elongate Device Systems and Methods”), which is incorporated by reference herein in its entirety.

In embodiments where the elongate device 602 and/or medical tool 626 are actuated by a teleoperational assembly (e.g., the manipulator assembly 502), the drive unit 604 may include drive inputs that removably couple to and receive power from drive elements, such as actuators, of the teleoperational assembly. In some examples, the elongate device 602 and/or medical tool 626 may include gripping features, manual actuators, or other components for manually controlling the motion of the elongate device 602 and/or medical tool 626. The elongate device 602 may be steerable or, alternatively, the elongate device 602 may be non-steerable with no integrated mechanism for operator control of the bending of distal end 618. In some examples, one or more channels 621 (which may also be referred to as lumens), through which medical tools 626 can be deployed and used at a target anatomical location, may be defined by the interior walls of the flexible body 616 of the elongate device 602.

In some examples, the medical instrument system 600 (e.g., the elongate device 602 or medical tool 626) may include a flexible bronchial instrument, such as a bronchoscope or bronchial catheter, for use in examination, diagnosis, biopsy, and/or treatment of a lung. The medical instrument system 600 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 630 may be sent to the navigation system 632, where the information may be combined with information from the visualization system 631 and/or pre-operatively obtained models to provide the physician, clinician, surgeon, or other operator with real-time position information. In some examples, the real-time position information may be displayed on the display system 510 for use in the control of the medical instrument system 600. In some examples, the navigation system 632 may utilize the position information as feedback for positioning medical instrument system 600. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

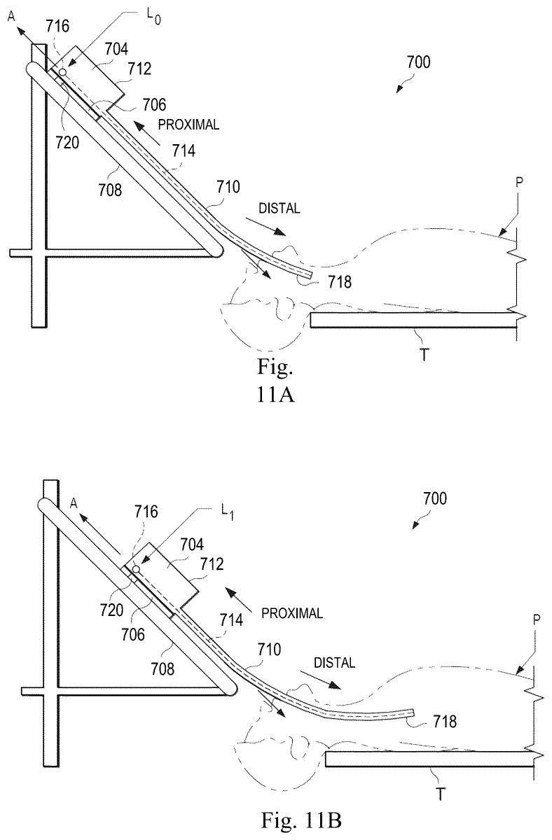

FIGS. 11A and 11B are simplified diagrams of side views of a patient coordinate space including a medical instrument mounted on an insertion assembly according to some embodiments. As shown in FIGS. 11A and 11B, a surgical environment 700 may include a patient P positioned on the patient table T. Patient P may be stationary within the surgical environment 700 in the sense that gross patient movement is limited by sedation, restraint, and/or other means. Cyclic anatomic motion, including respiration and cardiac motion, of patient P may continue. Within surgical environment 700, a medical instrument 704 is used to perform a medical procedure which may include, for example, surgery, biopsy, ablation, illumination, irrigation, suction, or electroporation. The medical instrument 704 may also be used to perform other types of procedures, such as a registration procedure to associate the position, orientation, and/or pose data captured by the sensor system 508 to a desired (e.g., anatomical or system) reference frame. The medical instrument 704 may be, for example, the medical instrument 504. In some examples, the medical instrument 704 may include an elongate device 710 (e.g., a catheter) coupled to an instrument body 712. Elongate device 710 includes one or more channels sized and shaped to receive a medical tool.

Elongate device 710 may also include one or more sensors (e.g., components of the sensor system 508). In some examples, a shape sensor 714 may be fixed at a proximal point 716 on the instrument body 712. The proximal point 716 of the shape sensor 714 may be movable with the instrument body 712, and the location of the proximal point 716 with respect to a desired reference frame may be known (e.g., via a tracking sensor or other tracking device). The shape sensor 714 may measure a shape from the proximal point 716 to another point, such as a distal end 718 of the elongate device 710. The shape sensor 714 may be aligned with the elongate device 710 (e.g., provided within an interior channel or mounted externally). In some examples, the shape sensor 714 may optical fibers used to generate shape information for the elongate device 710.

In some examples, position sensors (e.g., EM sensors) may be incorporated into the medical instrument 704. A series of position sensors may be positioned along the flexible elongate device 710 and used for shape sensing. Position sensors may be used alternatively to the shape sensor 714 or with the shape sensor 714, such as to improve the accuracy of shape sensing or to verify shape information.

Elongate device 710 may house cables, linkages, or other steering controls that extend between the instrument body 712 and the distal end 718 to controllably bend the distal end 718. In some examples, at least four cables are used to provide independent up-down steering to control a pitch of distal end 718 and left-right steering to control a yaw of distal end 718. The instrument body 712 may include drive inputs that removably couple to and receive power from drive elements, such as actuators, of a manipulator assembly.

The instrument body 712 may be coupled to an instrument carriage 706. The instrument carriage 706 may be mounted to an insertion stage 708 that is fixed within the surgical environment 700. Alternatively, the insertion stage 708 may be movable but have a known location (e.g., via a tracking sensor or other tracking device) within surgical environment 700. Instrument carriage 706 may be a component of a manipulator assembly (e.g., manipulator assembly 502) that couples to the medical instrument 704 to control insertion motion (e.g., motion along an insertion axis A) and/or motion of the distal end 718 of the elongate device 710 in multiple directions, such as yaw, pitch, and/or roll. The instrument carriage 706 or insertion stage 708 may include actuators, such as servomotors, that control motion of instrument carriage 706 along the insertion stage 708.

A sensor device 720, which may be a component of the sensor system 508, may provide information about the position of the instrument body 712 as it moves relative to the insertion stage 708 along the insertion axis A. The sensor device 720 may include one or more resolvers, encoders, potentiometers, and/or other sensors that measure the rotation and/or orientation of the actuators controlling the motion of the instrument carriage 706, thus indicating the motion of the instrument body 712. In some embodiments, the insertion stage 708 has a linear track as shown in FIGS. 11A and 11B. In some embodiments, the insertion stage 708 may have curved track or have a combination of curved and linear track sections.

FIG. 11A shows the instrument body 712 and the instrument carriage 706 in a retracted position along the insertion stage 708. In this retracted position, the proximal point 716 is at a position L0 on the insertion axis A. The location of the proximal point 716 may be set to a zero value and/or other reference value to provide a base reference (e.g., corresponding to the origin of a desired reference frame) to describe the position of the instrument carriage 706 along the insertion stage 708. In the retracted position, the distal end 718 of the elongate device 710 may be positioned just inside an entry orifice of patient P. Also in the retracted position, the data captured by the sensor device 720 may be set to a zero value and/or other reference value (e.g., I=0). In FIG. 11B, the instrument body 712 and the instrument carriage 706 have advanced along the linear track of insertion stage 708, and the distal end 718 of the elongate device 710 has advanced into patient P. In this advanced position, the proximal point 716 is at a position L1 on the insertion axis A. In some examples, the rotation and/or orientation of the actuators measured by the sensor device 720 indicating movement of the instrument carriage 706 along the insertion stage 708 and/or one or more position sensors associated with instrument carriage 706 and/or the insertion stage 708 may be used to determine the position L1 of the proximal point 716 relative to the position L0. In some examples, the position L1 may further be used as an indicator of the distance or insertion depth to which the distal end 718 of the elongate device 710 is inserted into the passageway(s) of the anatomy of patient P.

One or more components of the embodiments discussed in this disclosure, such as control system 110, 512, may be implemented in software for execution on one or more processors of a computer system. The software may include code that, when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

### Examples

The graphs of FIGS. 12-18 show differences in temperature measurements from two thermocouples placed between the case and heatsink of a microwave generator at various forward power level settings and various reverse/reflected power levels.

FIG. 12 shows differences in temperature measurements at various forward power level settings with a well-matched load (i.e., insignificant reverse power). One thermocouple is positioned nearest to the microwave generator's output stage transistor (labeled “Amplifier”) and the other thermocouple is positioned nearest to the microwave generator's isolator termination resistor (labeled “Isolator”). As shown, the temperature difference (Amplifier-Isolator) in FIG. 12 quickly rises to ˜5° C. and then levels off at a thermally stable level ˜4° C. It is believed that the general concept of a positive temperature difference between an Amplifier and Isolator at a matched load condition will apply to other configurations of microwave generator, cooling solution, and temperature sensor.

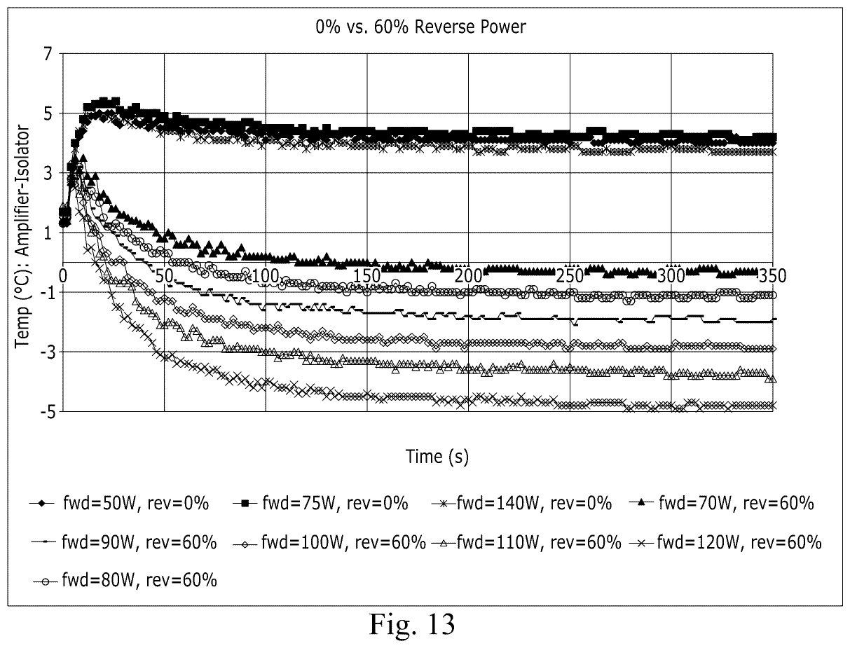

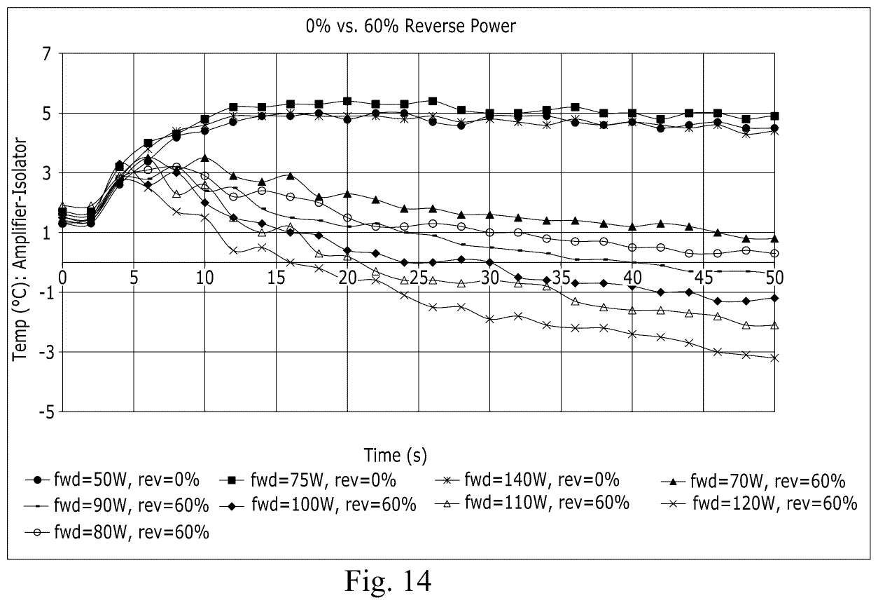

FIG. 13 shows the same differences in temperature measurements as FIG. 12 with the addition of test cases having 60% reflection. FIG. 14 shows the first 50 seconds of the measurements of FIG. 13 for clarity during initial warmup. As shown in FIGS. 13 and 14, and the following figures, the tests having reflections begin in a similar temperature rise as tests with matched loads (i.e., no reflection), however they quickly diverge as the reverse power heats the isolator's termination resistor. In less than 30 seconds, the level and trajectory of the temperature difference reveals the presence of reverse power.

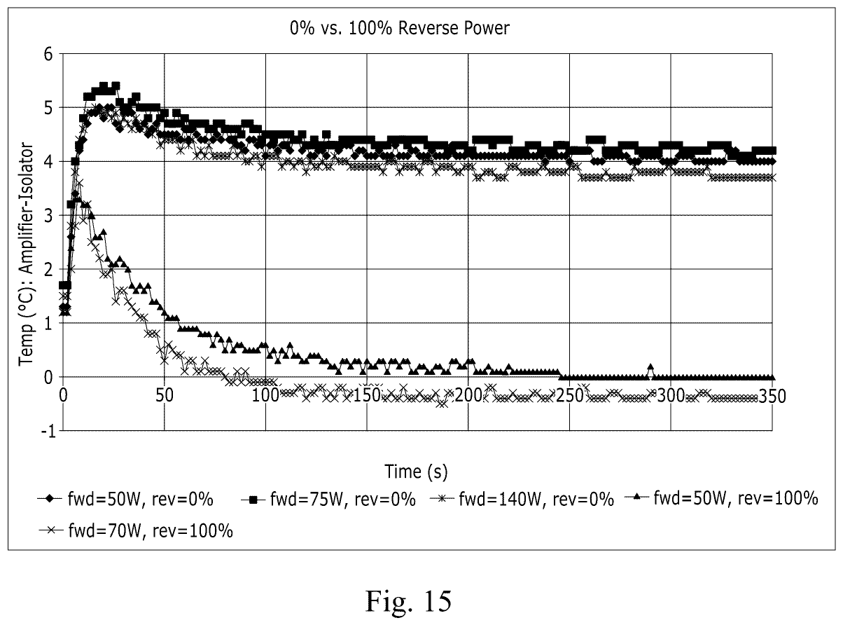

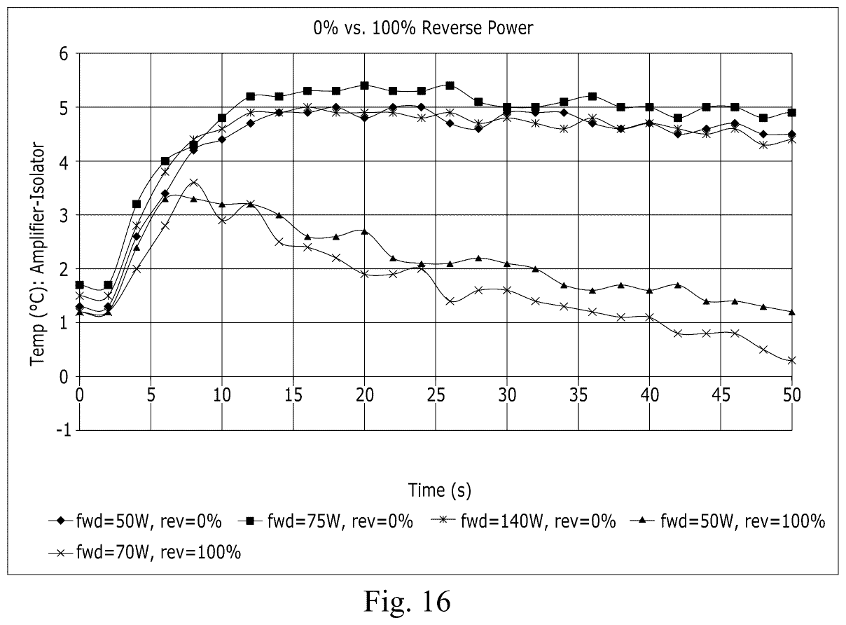

FIG. 15 shows the same differences in temperature measurements as FIG. 12 with the addition of test cases having 100% reflection. FIG. 16 shows the first 50 seconds of the measurements of FIG. 15 for clarity during initial warmup. While the range of forward power tests with 100% reflection are limited to 70 W to avoid overheating the microwave generator, FIGS. 15 and 16 show a faster divergence of the measurements due to a higher percentage of forward power being reflected by a larger impedance mismatch (e.g., open or short circuit).

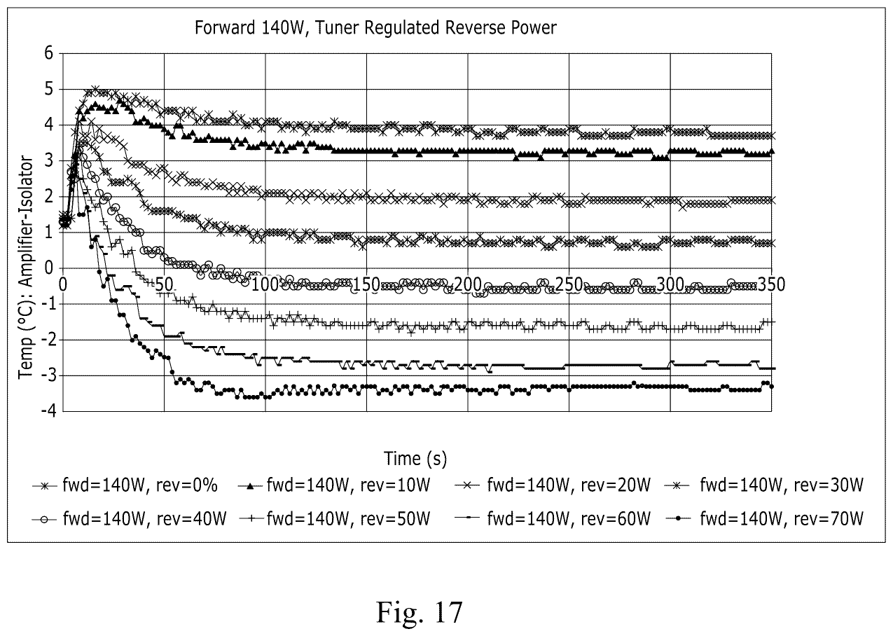

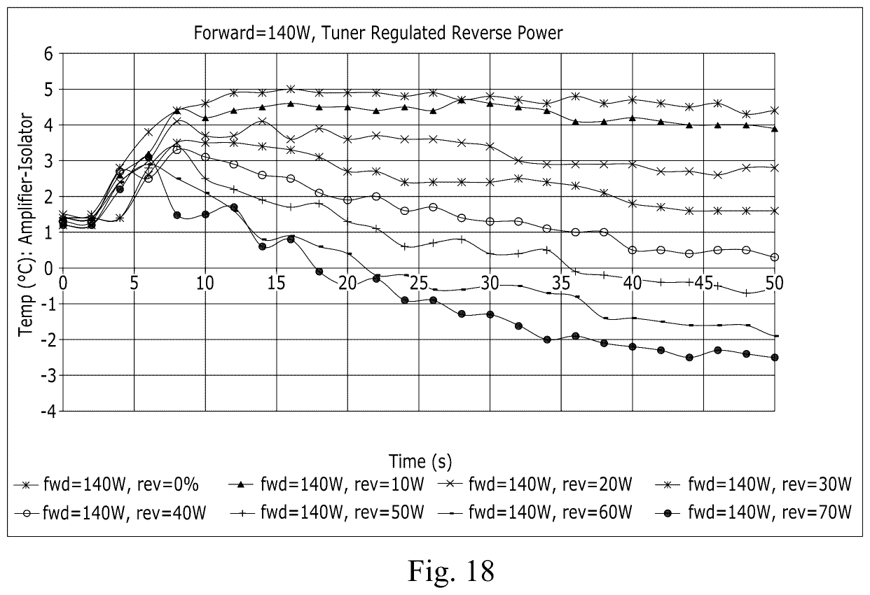

FIGS. 17 and 18 show differences in temperature measurements for a commonly maximum allowed microwave ablation forward power (140 W) at various reverse levels. The reverse level range is limited to avoid overheating the microwave generator; however, as shown, the data confirms that the larger the reflection, the more the temperature will rise at the isolator's termination resistor as compared to the microwave generator's output stage. The benefit of this property is that detection becomes easier and faster when a smaller fraction of output power is delivered to the load due to a high reflection. In such cases, it is easy to catch an inoperable microwave sensor for reverse power and thereby avoid potentially relying on a silently failed sensor. It is believed that the level of precision may be improved upon with better temperature sensing.

The terms “generally,” “approximately,” and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1. A microwave energy system comprising:
a microwave energy source receiving power supplied from a power supply;
an ablation probe coupled to the microwave energy source to receive a forward power from the microwave energy source; and
a control system configured to:
measure the power supplied to the microwave energy source by the power supply;
determine lost power due to inefficiency of the microwave energy source; and
determine the forward power from the microwave energy source by subtracting the lost power from the power supplied to the microwave energy source.

2. The microwave energy system of claim 1, further comprising a forward microwave power sensor configured to measure the forward power, wherein the control system is configured to determine the forward power independent of the forward microwave power sensor.

3. The microwave energy system of claim 2, wherein the control system is further configured to determine a state of the forward microwave power sensor by comparing the forward power to a measurement performed by the forward microwave power sensor.

4. The microwave energy system of claim 1, further comprising a temperature sensor configured to measure a temperature associated with the microwave energy source, and wherein the inefficiency of the microwave energy source is determined based on the temperature.

5. The microwave energy system of claim 4, wherein the microwave energy source comprises an output stage transistor, wherein the temperature sensor is associated with the output stage transistor.

6. The microwave energy system of claim 5, wherein the microwave energy source further comprises a termination resistor and a circuit board; the temperature sensor, the output stage transistor, and the termination resistor are mounted to the circuit board of the microwave energy source; and the temperature sensor is mounted to the circuit board on a side of the output stage transistor opposite of the termination resistor.

7. The microwave energy system of claim 5, wherein the microwave energy source further comprises an outer housing and a circuit board, the output stage transistor being mounted to the circuit board; and the temperature sensor comprises an external temperature sensor extending through the housing to be disposed adjacent to a side of the circuit board opposite the output stage transistor.

8. The microwave energy system of claim 5, wherein the control system is further configured to:
determine an expected temperature of output stage transistor;
determine an actual temperature associated with the output stage transistor based on data from the temperature sensor; and
compare the actual temperature with the expected temperature.

9. The microwave energy system of claim 5, wherein the control system is further configured to track a temperature of the output stage transistor over time to detect a drift in the inefficiency of the microwave energy source.

10. The microwave energy system of claim 9, wherein the control system is configured to adjust the inefficiency determination of the microwave energy source in response to detecting the drift.

11. The microwave energy system of claim 4, wherein the temperature sensor comprises a first temperature sensor configured to measure a first temperature associated with a forward power heat source of the microwave energy source; and further comprising a second temperature sensor configured to measure a second temperature associated with a termination point for reverse power of the microwave energy source; wherein the control system is configured to determine reverse power for the microwave energy source based on a temperature difference between the first and second temperatures.

12. The microwave energy system of claim 11, wherein the first temperature sensor is disposed adjacent to an output stage of the microwave energy source and the second temperature sensor is disposed adjacent to a termination point of the microwave energy source.

13. The microwave energy system of claim 12, wherein the first temperature sensor is disposed at a location that is minimally heated by the termination point of the microwave energy source and the second temperature sensor is disposed at a location that is minimally heated by the output stage of the microwave energy source.

14. The microwave energy system of claim 1, wherein the control system is further configured to adjust the microwave energy source based on the forward power determination.

15. A method for power detection in an ablation microwave energy system, the method comprising:
measuring power supplied to a microwave energy source by a power supply;
determining lost power due to inefficiency of the microwave energy source; and
determining forward power sent to an ablation probe from the microwave energy source by subtracting the lost power from the power supplied to the microwave energy source.

16. The method of claim 15, further comprising determining the forward power independent of a forward microwave power sensor configured to measure the forward power.

17. The method of claim 16, further comprising determining a state of the forward microwave power sensor by comparing the forward power to a measurement performed by the forward microwave power sensor.

18. The method of claim 15, further comprising measuring a temperature associated with the microwave energy source with a temperature sensor, the inefficiency of the microwave energy source being determined based on the temperature.

19. The method of claim 18, wherein measuring the temperature associated with the microwave energy source comprises measuring a temperature of an output stage transistor.

20. The method of claim 19, further comprising:
determining an expected temperature of the output stage transistor;
determining an actual temperature associated with the output stage transistor based on data from the temperature sensor; and
comparing the actual temperature with the expected temperature.

21. The method of claim 19, further comprising tracking a temperature of the output stage transistor over time to detect a drift in the inefficiency of the microwave energy source.

22. The method of claim 21, further comprising adjusting the inefficiency determination of the microwave energy source in response to detecting the drift.

23. The method of claim 18, wherein measuring the temperature associated with the microwave energy source with the temperature sensor comprises measuring a first temperature associated with a forward power heat source of the microwave energy source with a first temperature sensor; and further comprising:
measuring a second temperature associated with a termination point for reverse power of the microwave energy source with a second temperature sensor; and
determining reverse power for the microwave energy source based on a temperature difference between the first and second temperatures.

24. The method of claim 23, wherein the first temperature sensor is disposed adjacent to an output stage of the microwave energy source and the second temperature sensor is disposed adjacent to a termination point of the microwave energy source.

25. The method of claim 15, further comprising adjusting the microwave energy source based on the forward power determination.

26. A non-transitory computer readable medium having instructions stored thereon that, when executed by a computing device, cause the computing device to:
measure power supplied to a microwave energy source by a power supply;
determine lost power due to inefficiency of the microwave energy source; and
determine forward power sent to an ablation probe from the microwave energy source by subtracting the lost power from the power supplied to the microwave energy source.

27. The non-transitory computer readable medium of claim 26, wherein the instructions, when executed, further cause the computing device to:
determine the forward power independent of a forward microwave power sensor configured to measure the forward power.

28. (canceled)

29. The non-transitory computer readable medium of any one of claim 26, wherein the instructions, when executed, further cause the computing device to:
measure a temperature associated with the microwave energy source with a temperature sensor, the inefficiency of the microwave energy source being determined based on the temperature.

30.-33. (canceled)

34. The non-transitory computer readable medium of claim 29, wherein measuring the temperature associated with the microwave energy source with the temperature sensor comprises measuring a first temperature associated with a forward power heat source of the microwave energy source with a first temperature sensor; and wherein the instructions, when executed, further cause the computing device to:
measure a second temperature associated with a termination point for reverse power of the microwave energy source with a second temperature sensor; and
determine reverse power for the microwave energy source based on a temperature difference between the first and second temperatures.

35. (canceled)

36. The non-transitory computer readable medium of claim 26, wherein the instructions, when executed, further cause the computing device to:
adjust the microwave energy source based on the forward power determination.

37.-100. (canceled)
